(19) United States
(12) Patent Application Publication
(10) Pub. No.: US 2001/0009912 A1
(43) Pub. Date: Jul. 26, 2001

(54) NEW DERIVATIVES OF PYRIDIL PIPERAZINE OR PYRIDAZINYL PIPERAZYL, PROCESS FOR PRODUCTION THEREOF AND MEDICAMENTS CONTAINING THESE COMPOUNDS

(76) Inventors: CHRISTOS TSAKLAKIDIS, WEINHEIM (DE); ALFRED MERTENS, SCHRIESHEIM (DE); GERD ZIMMERMANN, MANNHEIM (DE); WOLFGANG SCHAFER, MANNHEIM (DE); LIESEL DORGE, MANNHEIM (DE)

Correspondence Address: NIKAIDO MARMELSTEIN MURRAY & ORAM, METROPOLITAN SQUARE, 655 FIFTEENTH STREET NW, SUITE 330 G STREET LOBBY, WASHINGTON, DC, 200055701

(21) Appl. No.: 09/257,205

(22) Filed: Feb. 25, 1999

Related U.S. Application Data
(63) Continuation of application No. 08/875,988, filed on Aug. 11, 1997, now abandoned.

(30) Foreign Application Priority Data
Feb. 10, 1995 (DE) 195 04 329.4

(51) Int. Cl. A61K 31/497; C07D 403/00; C07D 401/00

(52) U.S. Cl. 514/252.14; 544/360; 544/364

(57) ABSTRACT

Compounds of formula I

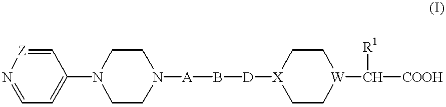

in which

R¹ denotes hydrogen, lower alkyl, lower alkenyl, cycloalkyl, cycloalkenyl, an optionally substituted monocyclic or bicyclic aryl, an optionally substituted hetaryl, an optionally substituted arylalkyl or one of the groups

—OR², —NR³R⁴,

W denotes nitrogen or

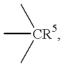

X, Z independently of one another denote nitrogen or

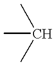

CH and in the case that W denotes a nitrogen atom, X must be the

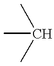

group,

A denotes a valency dash or a carbonyl group,

B denotes a valency dash or a C₁-C₆ alkylene chain optionally substituted once or several times by lower alkyl or an OR² group,

D denotes a valency dash and, in the case that X is a nitrogen atom, can also be a carbonyl group in which case A, B and D may not simultaneously denote a valency dash,

R² denotes hydrogen, lower alkyl or arylalkyl,

R³ and R⁴ independently of one another denote hydrogen or lower alkyl or together with the nitrogen atom to which they are bound form a five to six-membered heterocyclic ring,

R⁵ denotes hydrogen or a group OR²,

processes for the production thereof as well as pharmaceutical agents containing these compounds for the treatment of diseases which are the result of thrombo-embolic events.

It is known that compounds which carry a basic and an acidic group are capable of inhibiting the aggregation of blood platelets when the basic and acidic group in the compounds are at a very specific distance from one another (Drugs of the Future 19 (8), 757 (1994). Compounds with an anti-aggregatory action on blood platelets are described in the patent specifications WO 93/14077, EP-A 0 537 980, EP-A 0 542 363, WO 94/22834 and WO 94/22835.

The present invention concerns new pyridyl- and pyridazinyl-piperazine derivatives, processes for the production thereof as well as pharmaceutical agents containing these substances.

It was now found that pyridine and pyridazine derivatives which additionally carry a carboxylic acid group effectively inhibit the aggregation of blood platelets and can thus be used to treat diseases that are due to thrombo-embolic events such as stroke, myocardial infarction or arterial occlusive diseases as well as inflammations, osteoporosis or tumour diseases.

The present invention concerns compounds of the general formula I

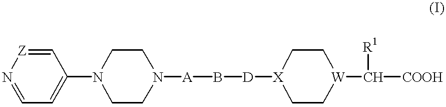

in which

R¹ denotes hydrogen, lower alkyl, lower alkenyl, cycloalkyl, cycloalkenyl, an optionally substituted monocyclic or bicyclic aryl, an optionally substituted hetaryl, an optionally substituted arylalkyl or one of the groups

—OR², —NR³R⁴,

W denotes nitrogen or

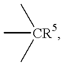

X, Z independently of one another denote nitrogen or

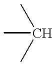

and in the case that W denotes a nitrogen atom, X must be the

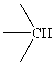

group,

A denotes a valency dash or a carbonyl group,

B denotes a valency dash or a C₁-C₆ alkylene chain optionally substituted once or several times by lower alkyl or an OR² group,

D denotes a valency dash and, in the case that X is a nitrogen atom, can also be a carbonyl group in which case A, B and D may not simultaneously denote a valency dash,

R² denotes hydrogen, lower alkyl or arylalkyl,

R³,R⁴ independently of one another denote hydrogen or lower alkyl or together with the nitrogen atom to which they are bound form a five to six-membered heterocyclic ring,

R⁵ denotes hydrogen or a group —OR²,

as well as pharmacologically acceptable salts thereof.

In all cases lower alkyl should represent a straight-chained or branched C₁-C₆ alkyl group such as e.g. methyl, ethyl, propyl, isopropyl, butyl, isobutyl, pentyl or hexyl in particular methyl, ethyl, propyl, isobutyl and pentyl.

Lower alkenyl denotes unsaturated residues with 3-6 carbon atoms such as allyl, but-2-enyl, hexa-2,4-dienyl but above all allyl.

Cycloalkyl denotes an optionally substituted 3-7-membered ring such as a cyclopropyl, cyclobutyl, cyclopentyl, cyclohexyl or cycloheptyl ring in particular a cyclopropyl, cyclopentyl and cyclohexyl ring. These cycloalkyl residues can be substituted once or twice by a C₁-C₆ alkyl group preferably a methyl, ethyl or isopropyl group as well as by hydroxy, methoxy, benzyloxy, amino, methylamino, dimethylamino or benzylamino groups or by chlorine or bromine.

Cycloalkenyl denotes an optionally substituted cyclopentenyl, cyclohexenyl or cycloheptenyl ring. These rings can be substituted once or twice by a C₁-C₆ alkyl group preferably a methyl, ethyl or isopropyl group as well as by chlorine, bromine or hydroxy, methoxy, benzyloxy, amino, methylamino, dimethylamino or benzylamino groups.

If the residues R³ and R⁴ form a heterocyclic ring together with the nitrogen atom to which they are bound then this is a saturated or unsaturated 5-6-membered ring such as a pyrrolidine, piperidine, morpholine pyrroline, piperidine or morpholine ring.

The carbocyclic and heterocyclic rings can be optionally substituted once or twice by C₁-C₆ alkyl groups, preferably a methyl, ethyl or isopropyl groups as well as by chlorine, bromine or hydroxy, methoxy, benzyloxy, amino, methylamino, dimethylamino or benzylamino groups.

Aryl usually denotes a phenyl residue which is optionally substituted once or several times. Hetaryl usually denotes a pyridine, pyridazine, pyrrole, thiophene, furan or imidazole ring which is substituted once or several times.

Bicyclic aryl usually denotes an indane or naphthalene residue which is optionally substituted once or several times, preferably a naphthalene residue. Aryl, bicyclic aryl and hetaryl residues can be optionally substituted once or several times by C₁-C₆ alkyl groups, preferably a methyl, ethyl or isopropyl group as well as by chlorine, bromine, fluorine or hydroxy, alkoxy such as e.g. methoxy, benzyloxy, acetyloxy, carboxy, ethoxycarbonyl, aminocarbonyl, methylaminocarbonyl, dimethylaminocarbonyl, cyano, amino, methylamino, dimethylamino, benzylamino, acetylamino, benzoylamino and amidine groups.

Arylalkyl usually denotes an unsubstituted or once or several-fold substituted benzyl, phenethyl, phenylpropyl, phenylbutyl or phenylpentyl residue, preferably a benzyl, phenethyl or phenylpentyl residue. C₁-C₆ alkyl residues, preferably a methyl, ethyl or isopropyl group as well as chlorine, bromine, fluorine or hydroxy, methoxy, benzyloxy, acetyloxy, carboxy, ethoxycarbonyl, aminocarbonyl, methylaminocarbonyl, dimethylaminocarbonyl, cyano, amino, methylamino, dimethylamino, benzylamino, acetylamino, benzoylamino and amidino groups come into consideration as substituents.

Compounds of the general formula I contain at least one asymmetric carbon atom and therefore optically active compounds of the general formula I are also a subject matter of the present application. Furthermore conformation isomers of compounds of the general formula I which may occur are also a subject matter of the present application.

Preferred compounds are compounds of formula I in which the group A-B-D represents a group (CH₂)₁₋₃ or CO—(CH₂)₁₋₃ and Z, X, W and R¹ have the stated meaning.

Compounds of formula I are additionally preferred in which the ring

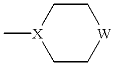

represents a 1,4-cyclohexylidenyl or a 1,4-piperidinyl ring and Z, A-B-D and R¹ have the meaning stated above.

Compounds of formula I are especially preferred in which A-B-D represents ethylene or carbonylethylene group and the ring

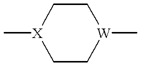

represents a 1,4-cyclohexylidenyl ring and Z and R¹ have the stated meaning.

Compounds of the general formula I are produced according to well-known processes by hydrolyzing an ester of the general formula II

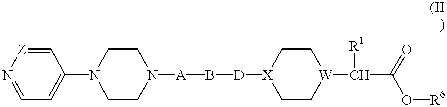

in which R¹, A, B, D, W, X, and Z have the meanings stated above and R⁶ denotes a methyl, ethyl, tert.-butyl or benzyl residue.

Compounds of the general formula II are new and are prepared according to well-known processes and preferably by the following:

a) in the case that W denotes nitrogen, a compound of the general formula III

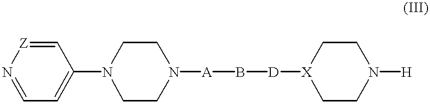

in which A, B, D and Z have the meanings stated above is reacted with a compound of the general formula IV

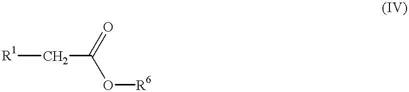

in which R¹ and R⁶ have the meanings stated above and L denotes a leaving group such as Hal or O—SO₂₋R⁷ in which Hal can be chloride, bromide or iodide and R⁷ should be methyl, phenyl, p-methylphenyl or p-nitrophenyl,

b) in the case that W denotes a group CR⁵ and R⁵ denotes a group OR², a ketone of the general formula V

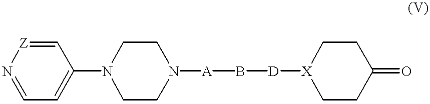

in which A, B, D, X and Z have the meanings stated above is reacted with a carboxylic acid ester of the general formula VI

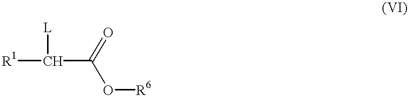

in which R¹ and R⁶ have the meanings stated above and the hydroxyl group of the 2-hydroxy ester which forms in this process of the general formula VII

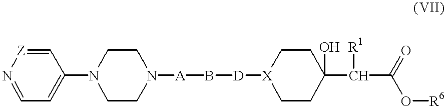

in which R¹, R⁶, X, Z, A, B and D have the meanings stated above is alkylated if desired with an alkylating agent of the general formula VIII

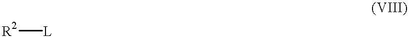

in which R² and L have the meanings stated above or

c) in the case that W denotes a CH group, a compound of the general formula VI is alkylated with a compound of the general formula IX

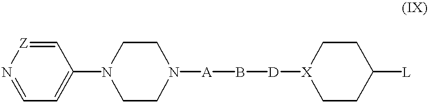

in which A, B, D, X, Z and L have the meanings stated above or

d) the olefinic double bond of a compound of the general formula X

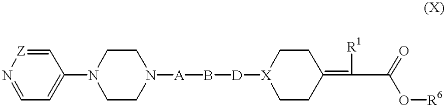

in which A, B, D, R¹, R⁶, X, and Z have the meanings stated above is catalytically hydrogenated.

e) in the case that A and D each denote a carbonyl group, a dicarboxylic acid derivative of the general formula XI

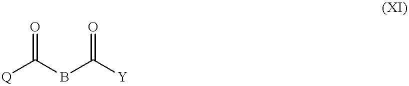

in which B has the meaning stated above and Y and Q independently of each other denote hydrogen, the group OR² in which R² has the meanings stated above or denotes a halogen such as chlorine or bromine is reacted successively with an amine of the general formula XII

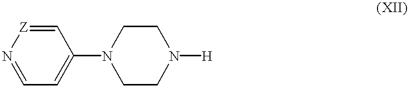

in which Z has the meaning stated above and with an amine of the general formula XIII

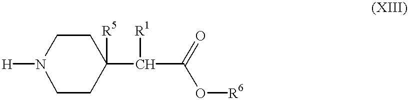

in which R¹, R⁵ and R⁶ have the meanings stated above.

Compounds of the general formula III are new and are produced according to well-known processes and preferably by reacting a compound of the general formula XII with a compound of the general formula XIV

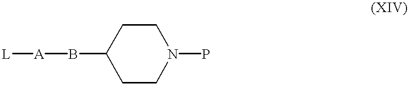

in which A, B and L have the meanings stated above and P denotes a protecting group for amines such as acetyl, tert.-butyloxycarbonyl, benzyl or benzyloxycarbonyl and subsequently the protecting group P is removed from the product that is formed.

Compounds of the general formula IV are produced in such a way that in the case that L=Hal, a compound of the general formula VI is halogenated according to processes known in the literature, or in the case that L in formula IV denotes an O—SO₂—R⁷ group, the hydroxyl group of a compound of the general formula XV

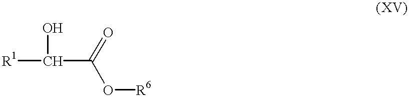

in which R¹ and R⁶ have the meanings stated above is converted into the corresponding sulfonic acid ester.

Compounds of the general formula V are new and are usually obtained by cleaving the ketal group of a compound of the general formula XVI

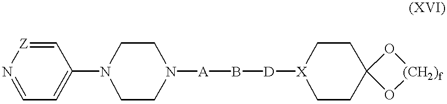

in which A, B, D, X, and Z have the meanings stated above and f=2,3.

Compounds of the general formula VIII can be obtained commercially in the case that L=Hal; in the case that L denotes an O—SO₂—R⁷ group, the hydroxyl group of commercially available alcohols of the general formula (XVII)

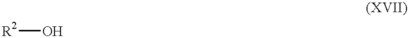

in which R² has the meanings stated above is converted into the corresponding sulfonic acid ester.

Compounds of the general formula IX are new and if L denotes an O—SO₂—R⁷ group they are produced by converting the hydroxyl group of a compound of the general formula XVIII,

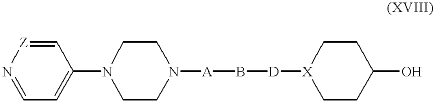

in which A, B, D, X, and Z have the meanings stated above, into the corresponding sulfonic acid ester; in the case that L=Hal, the hydroxyl group of a compound of the general formula XVIII is nucleophilically substituted by halogen according to processes known from the literature.

Compounds of the general formula X are new and are produced in a well-known manner by subjecting a ketone of the general formula V to a Wittig reaction with a phosphorane of the general formula XIX,

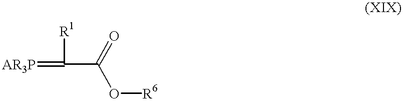

in which R¹ and R⁶ have the meanings stated above and Ar denotes an aryl within the sense of the definition for aryl given above, or the ketone of formula V is subjected to a Horner-Emmons reaction with a phosphonoacetic acid ester of the general formula XX

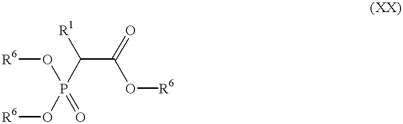

in which R¹ and R⁶ have the meanings stated above.

Compounds of the general formula XI are commercially available.

Compounds of the general formula XII are prepared by reacting 1-benzylpiperazine with 4-chloropyridine or 4-chloropyrazine.

Compounds of the general formula XIII are produced in such a way that

a) in the case that R⁵ denotes hydrogen, the double bond of a compound of the general formula XXI

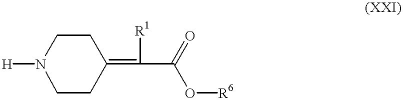

in which R¹ and R⁶ have the meanings stated above is catalytically hydrogenated and

b) in the case that R⁵ denotes the group OR², a compound of the general formula VI is reacted with 4-piperidone and the hydroxyl group of the 2-hydroxy ester of the general formula XXII that forms

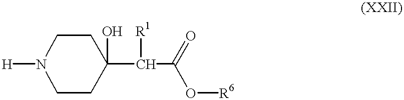

in which R¹ and R⁶ have the meanings stated above is alkylated with an alkylation agent of the general formula VIII.

Compounds of the general formula XIV are produced according to well-known processes in such a way that

a) in the case that A denotes a valency dash, the hydroxyl group of an alcohol of the general formula XXIII

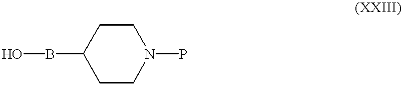

in which B and P have the meanings stated above is appropriately halogenated or sulfonated and

b) in the case that A denotes a carbonyl group, a carboxylic acid of the general formula XXIV

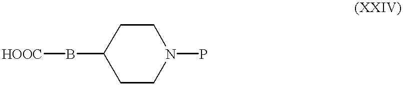

in which B and P have the meanings stated above is converted into the corresponding carboxylic acid halogenide.

Compounds of the general formula XV can be obtained according to literature methods by oxidizing the corresponding compounds of the general formula VI.

Compounds of the general formula XVI are prepared in such a way that a compound of the general formula XII is reacted with a compound of the general formula XXV

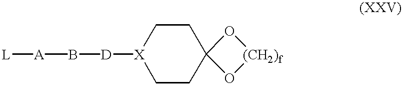

in which L, A, B, D, X and f have the meanings stated above.

Alcohols of the general formula XVII are commercially available.

Compounds of the general formula XVIII are prepared by reducing the carbonyl group of a compound of the general formula V.

Some of the compounds of the general formula XIX are commercially available (Aldrich Chemie GmbH and Co. KG) and are obtained in special cases according to known processes by reacting a 2-halogen-carboxylic acid derivative of the general formula IV with a triarylphosphine of the general formula XXVI

AR₃P (XXVI)

in which Ar has the meanings stated above.

Some of the compounds of the general formula XX are commercially available (Aldrich Chemie GmbH and Co. KG) and in special cases are obtained according to known processes by the Arbuzov reaction between a 2-halogen-carboxylic acid derivative of formula IV and a trialkylphosphite of the general formula XXVII

(OR⁶)₃P (XXVII)

in which R⁶ has the meanings stated above.

Compounds of the general formula XXI are prepared in such a way that a 4-piperidone of the general formula XXVIII

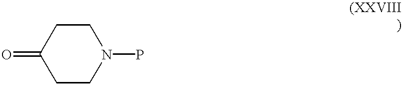

in which P has the meanings stated above is reacted with a compound of formula XIX or of formula XX and the protecting group P is removed from the product that forms.

Compounds of the general formula XXIII are obtained by reducing the carboxyl group of a compound of formula XXIV.

Some of the compounds of the general formula XXIV are commercially available or are described in the literature (Ishihara, Chem. Pharm. Bull. 41, 529 (1993); Merck and. Co. EP 478362).

Compounds of the general formula XXV are prepared according to known processes in such a way that

a) in the case that X=CH and A denotes a valency dash, the hydroxyl group of an alcohol of the general formula XXIX

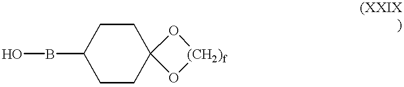

in which B and f have the meanings stated above is converted into a halogen or a sulfonic acid ester,

b) in the case that X CH and A denotes a carbonyl group, a carboxylic acid of the general formula XXX

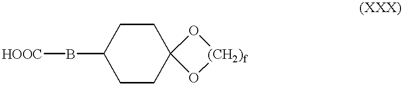

in which B and f have the meanings stated above is converted into the corresponding carboxylic acid halogenide and

c) in the case that X=N, a piperidone derivative of the general formula XXXI

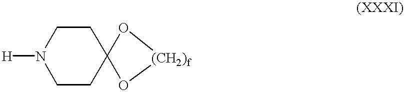

in which f has the meanings stated above is reacted with a compound of the general formula XXXII

L-A-B-D-L (XXXII)

in which L, A, B and D have the meanings stated above.

Triarylphosphines of formula XXVI, trialkylphosphites of formula XXVII and 4-piperidones of formula XXVIII are commercially available.

Compounds of the general formula XXIX are obtained by reducing the carbonyl group of a compound of formula XXX.

Compounds of the general formula XXX are obtained by hydrolyzing an ester of the general formula XXXIII

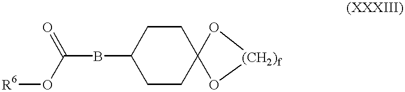

in which R⁶, B and f have the meanings stated above.

Compounds of the general formula XXXI are commercially available.

Compounds of the general formula XXXII are commercially available in the case that L=Hal; in the case that L denotes a sulfonic acid residue, commercially available 1-omegadiols are sulfonated appropriately; in the case that A or D denote a carbonyl group and L=Hal, omega-halogen-carboxylic acids which are commercially available are converted into the respective carboxylic acid halogenide and in the case that A and D denote a carbonyl group these are compounds of the general formula XI.

Some of the compounds of the general formula XXXIII can be obtained commercially and are in special cases obtained by catalytic hydrogenation of the double bond of a compound of the general formula XXXIV

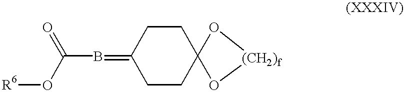

in which B, R⁶ and f have the meanings stated above.

Compounds of the general formula XXXIV can be obtained according to well-known processes by reacting a 1,4-cyclohexanedione derivative of the general formula XXXV

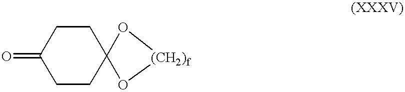

in which f has the meanings stated above with a phosphorane of the general formula XXXVI

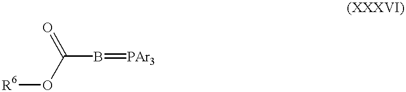

in which R⁶, B and Ar have the meanings stated above.

Compounds of the general formula XXXVI are obtained in such a way that a triarylphosphine of formula XXVI is reacted with a commercially available omega-halogen-carboxylic acid ester.

An ester of the general formula II or of formula XXXIII is hydrolyzed to the corresponding carboxylic acid of the general formula I or XXX according to the usual methods by treating a carboxylic acid ester of the general formula II in water or in a mixture of water, tetrahydrofuran, dioxane, methanol or ethanol, preferably in a mixture of water/tetrahydrofuran, with a hydroxide such as sodium, potassium or lithium hydroxide, preferably sodium or lithium hydroxide, or with an acid such as hydrochloric acid, sulphuric acid or trifluoroacetic acid, preferably trifluoroacetic acid, and at temperatures between room temperature and 80° C., preferably at room temperature.

Reaction of a compound of the general formula III with a compound of formula IV or a compound of formula VII with a compound of formula VIII or a compound of formula VI with a compound of formula IX or a compound of formula XXII with a compound of formula VIII or a compound of formula XII with a compound of formula XIV or a compound of formula XII with a compound of formula XXV or a compound of formula XXXI with a compound of formula XXXII or reaction of 4-chloropyridine or 4-chloropyridazine with 1-benzylpiperazine is usually carried out in an aprotic solvent such as toluene, tetrahydrofuran, diethyl ether or dimethylformamide, preferably dimethylformamide or tetrahydrofuran using a base such as potassium hydride, sodium hydride, potassium carbonate or sodium bicarbonate, preferably sodium hydride or potassium carbonate, and at temperatures between room temperatures and 180° C., preferably at 120° C.

A ketone of the general formula V is reacted with an ester of the general formula VI, or 4-piperidone is reacted with an ester of formula VI under the conditions of an aldol reaction in a solvent such as methanol, ethanol, toluene, tetrahydrofuran, diethyl ether or dimethylformamide, preferably tetrahydrofuran or dimethylformamide, using a base such as sodium or potassium methylate or sodium or potassium ethylate, sodium hydride, potassium hydride, lithium diisopropylamide, potassium hexamethyl disilazide, preferably sodium hydride or lithium diisopropylamide and at temperatures between −78° C. and 90° C., however, preferably between −78° C. and room temperature.

The catalytic hydrogenation of the olefinic double bond of a compound of the general formula X or of formula XXI or of formula XXXIV is carried out analogously to processes known from the literature (A. Nose, Chem. Pharm. Bull. 38, 2097 (1990); Tamura M. Bull. Chem. Soc. Jpn. 53, 561 (1980); Liu H.-J., Synth. Commun. 15, 965 (1985); Chido N., J. Chem. Soc. Chem. Commun. 994 (1990); Büchi G., J. Amer. Chem. Soc. 89, 6745 (1967); Ernst I., Coll. Czech, Chem. Comm. 24, 3341 (1959); Johnson W. S., J. Amer. Chem. Soc. 79, 1995 (1957); Muchowski J. M., Can. J. Chem. 47, 857 (1969)).

The protecting group P can if necessary be removed from compounds which carry the protecting group P and which are described in or encompassed by this patent specification by treating a compound carrying the protecting group P with aqueous mineral acids or bases such as hydrochloric acid, sulfuric acid or trifluoroacetic acid or sodium hydroxide solution or potassium hydroxide solution or subjecting it to a catalytic hydrogenation such as e.g. with palladium/carbon/hydrogen.

A compound of the general formula VI or of formula XVIII or of formula XXIII or of formula XXIV or of formula XXIX or of formula XXX is halogenated by reacting it with molecular halogen (chlorine, bromine, iodine), preferably bromine, without a solvent or in an inert solvent such as methylene chloride, chloroform or carbon tetrachloride, preferably carbon tetrachloride, and with addition of red phosphorus, phosphorus trichloride or phosphorus tribromide and at a temperature between room temperature and 100° C., preferably at 90° C. (K. Stoh, Chem. Pharm. Bull. 34, 2078 (1986); H. J. Ziegler, Synthesis 1969, 39)). In addition, compounds of the general formula VI can be halogenated by metalation with a lithium amide such as lithium diisopropylamide in an aprotic solvent such as tetrahydrofuran and at a low temperature, preferably at -78° C., and subsequently reacting the compounds of the general formula XVI which are metalized in the a position with bromine, iodine, carbon tetrachloride or carbon tetrabromide (M. Hesse, Helv. Chem. Acta 72, 847 (1989) R. T. Arnold, J. Org. Chem. 43, 3687 (1978)) or with N-chlorosuccinimide or N-bromosuccinimide (W. Oppolzer, Tetrahedron Lett. 26, 5037 (1985).

The hydroxyl group of a compound of the general formula XV, XVII, XVIII, XXIII or XXIX is converted into a sulfonic acid ester according to the usual methods such as by condensation with a sulfonyl chloride such as methanesulfonyl chloride, benzenesulfonyl chloride, p-toluenesulfonyl chloride or p-nitrobenzenesulfonyl chloride, preferably methanesulfonyl chloride or p-toluenesulfonyl chloride, in an inert solvent such as methylene chloride, tetrahydrofuran or diethyl ether, preferably methylene chloride, using an auxiliary base such as trimethylamine or triethylamine or pyridine, preferably triethylamine, and at a temperature between 0° C. and room temperature.

Ketal cleavage of a ketal of the general formula XVI is carried out according to standard procedures in organic chemistry (ORGANIKUM; VEB “Deutscher Verlag der Wissenschaften”, Berlin 1977, page 486, 490).

The Wittig reaction between a ketone of the general formula V and a phosphorane of the general formula XIX, or a ketone of formula XXVIII and a phosphorane of the formula XIX, or a ketone of formula XXXV and a phosphorane of formula XXXVI is carried out according to known methods by reflux heating the reactants in an aprotic solvent such as benzene, toluene or xylol, preferably toluene.

The Horner-Emmons reaction between a ketone of the general formula V and a phosphonoacetic acid ester of the general formula XX, or a ketone of formula XXVIII and a phosphonoacetic acid ester of the formula XX is usually carried out in a solvent such as dimethylformamide, tetrahydrofuran, diethyl ether or 1,4-dioxane, preferably dimethylformamide or tetrahydrofuran, using a base such as sodium hydride, butyllithium, lithium diisopropylamide or sodium hexamethyl disilazide, preferably sodium hydride or lithium diisopropylamide, and at a temperature between −78° C. and 100° C. preferably, however, at −78° C. or room temperature.

Oxidation of a compound of the general formula VI to form a compound of the general formula XV is usually carried out in a solvent such as tetrahydrofuran by addition of a base such as lithium diisopropylamide or lithium-N-isopropyl-N-cyclohexylamide using an oxidizing agent such as an oxaziridine derivative, molybdenum peroxide or atmospheric oxygen and at temperatures between −78° C. and room temperature, preferably at 50° C. (C. Tamm, Tetrahedron Lett. 26, 203 (1985); F. A. Davis J. Org. Chem. 51, 2402 (1986); C. Wintoai Synth. Commun. 18, 2141 (1988)).

The reaction of a compound of the general formula IV with a triarylphosphine of the general formula XXVI, or of a compound of formula XXXVI with a triarylphosphine of formula XXVI is carried out analogously to methods known from the literature (Buddras J., Angew. Chem. 80, 535 (1968); Bestmann H. J. Angew. Chem. 77, 620, 651 (1965); Wittig G. Ber. Deutsch. Chem. Ges. 88, 1654 (1955)).

A compound of the general formula IV is usually reacted with a compound of the general formula XXVII without a solvent at temperatures between room temperature and 150° C., preferably at 130° C., with a reaction time between 30 min and 30 hours, preferably 18 hours.

A carboxylic acid derivative of the general formula XXIV or XXX is usually reduced to an alcohol of formula XXIII or XXIX in a solvent such as tetrahydrofuran or diethyl ether using a reducing agent such as lithium aluminium hydride and at a reaction temperature between 0° C. and the reflux temperature of the solvent used, preferably at 40° C.

Acylation of an amine of the general formula XII or of formula XIII or of formula XXXI with a carboxylic acid derivative of formula XI or of formula XXV or of formula XXXII or of formula XIV is usually carried out in a solvent such as methylene chloride, dimethylformamide or pyridine, preferably methylene chloride or pyridine, with addition of an auxiliary base such as triethylamine or 4-dimethylamino-pyridine and at a temperature between −10° C. and 50° C., but preferably at room temperature.

A ketone of the general formula V is usually reduced to an alcohol of formula XVIII in a solvent such as methanol, ethanol, tetrahydrofuran or diethyl ether, preferably methanol, using a reducing agent such as sodium borohydride, lithium borohydride or lithium aluminium hydride, preferably sodium borohydride, and at a temperature between −10° C. and +30° C., preferably at room temperature.

Alkali salts, ammonium salts, trifluoroacetates or hydrochlorides are used above all as pharmacologically acceptable salts which are usually produced for example by titrating the compounds with inorganic or organic bases or acids such as e.g. sodium or potassium bicarbonate, sodium hydroxide solution, potassium hydroxide solution, aqueous ammonia or amines such as trimethylamine or triethylamine, trifluoroacetic acid or hydrochloric acid. The salts are usually purified by precipitation from water/acetone.

The new substances of formula I and salts thereof according to the invention can be administered enterally or parenterally in a liquid or solid form. In this connection all the usual forms of administration come into consideration such as tablets, capsules, dragées, syrups, solutions, suspensions etc.. Water is preferably used as the injection medium which contains the usual additives for injection solutions such as stabilizing agents, solubilizers and buffers.

Such additives are for example tartrate and citrate buffer, ethanol, complexing agents (such as ethylene-diaminetetraacetic acid and non-toxic salts thereof), high molecular polymers (such as liquid polyethylene oxide) in order to regulate viscosity. Liquid carriers for injection solutions have to be sterile and are preferably dispensed into ampoules. Solid carriers are e.g. starch, lactose, mannitol, methylcellulose, talcum, highly dispersed silicic acids, higher molecular fatty acids (such as stearic acid), gelatine, agar-agar, calcium phosphate, magnesium stearate, animal and vegetable fats, solid high molecular polymers (such as polyethylene glycols); preparations that are suitable for oral application can optionally contain flavourings and sweeteners.

The dose can depend on various factors such as manner of administration, species, age and/or individual state of health. The doses to be administered daily are about 10-1000 mg/human, preferably 100-500 mg/human and can be taken once or several times.

Within the sense of the present invention the following pyridine and pyridazine derivatives are preferred in addition to the compounds mentioned in the examples and compounds derived by combination of all meanings of substituents mentioned in the claims:

1. [4-(4-Pyridin-4-yl-piperazin-1-ylmethyl)-cyclohexyl]-acetic acid

2. {4-[2(4-Pyridin-4-yl-piperazin-1-yl) -ethyl]-cyclohexyl} -acetic acid

3. {4-[3-(4-Pyridin-4-yl-piperazin-1-yl) -propyl]-cyclohexyl)-acetic acid

4. {4-[4-(4-Pyridin-4-yl-piperazin-1-yl) -butyl]-cyclohexyl} -acetic acid

5. {4-[6-(4-(Pyridin-4-yl-piperazin-1-yl) -hexyl]-cyclohexyl} -acetic acid

6. [4-(4-Pyridin-4-yl-piperazin-1-carbonyl)-cyclohexyl]-acetic acid

7. {4-[2-Oxo-2(4-pyridin-4-yl -piperazin-1-yl) -ethyl]-cyclohexyl} -acetic acid

8. {4-[3-Oxo-3-(4-pyridin-4-yl-piperazin-1-yl)-propyl] -cyclohexyl}-acetic acid

9. {4-[4-Oxo-4-(4-pyridin-4-yl-piperazin-1-yl)-butyl] -cyclohexyl}-acetic acid

10. {4-[6-Oxo-6-(4-pyridin-4-yl-piperazin-1-yl)-hexyl] -cyclohexyl}-acetic acid

11. {1-[2-Oxo-2-(4-pyridin-4-yl-piperazin-1-yl)-acetyl] -piperidin-4-yl}-acetic acid

12. {1-[4-Oxo-4-(4-pyridin-4-yl-piperazin-1-yl)-butyryl] -piperidin-4-yl}-acetic acid

13. {1-[5-Oxo-5-(4-pyridin-4-yl-piperazin-1-yl)-pentanoyl] -piperidin-4-yl}-acetic acid

14. {1-[6-Oxo-6-(4-pyridin-4-yl-piperazin-1-yl)-hexanoyl] -piperidin-4-yl}-acetic acid

15. {1-[8-Oxo-8-(4-pyridin-4-yl-piperazin-1-yl)-octanoyl] -piperidin-4-yl}-acetic acid

16. {1-[3-(4-Pyridin-4-yl-piperazin-1-yl)-propionyl] -piperidin-4-yl}-acetic acid

17. {1-[4-(4-Pyridin-4-yl-piperazin-1-yl)-butyryl] -piperidin)-4-yl}-acetic acid

18. {1-[5-(4-Pyridin-4-yl-piperazin-1-yl)-pentanoyl] -piperidin-4-yl}-acetic acid

19. {1-[2-(4-Pyridin-4-yl-piperazin-1-yl]-acetyl] -piperidin-4-yl}-acetic acid

20. [4-(4-pyridin-4-yl-piperazin-1-carbonyl)-piperidin-1-yl]-acetic acid

21. {4-[2-Oxo-2-(4-pyridin-4-yl-piperazin-1-yl)-ethyl] -piperidin-1-yl}-acetic acid

22. {4-[3-Oxo-3-(4-pyridin-4-yl-piperazin-1-yl)-propyl] -piperidin-1-yl}-acetic acid

23. {4-[5-Oxo-5-(4-pyridin-4-yl-piperazin-1-yl)-pentyl] -piperidin-1-yl}-acetic acid

24. {4-[7-Oxo-7-(4-pyridin-4-yl-piperazin-1-yl)heptyl)-piperidin-1-yl}-acetic acid

25. [1-Hydroxy-4-(4-pyridin-4-yl-piperazin-1-carbonyl)-cyclohexyl] -acetic acid

26. [1-Hydroxy-4-(4-pyridin-4-yl-piperazin-1-ylmethyl)-cyclohexyl] -acetic acid

27. {1-Hydroxy-4-[2-(4-pyridin-4-yl-piperazin-1-yl)-ethyl] -cyclohexyl}-acetic acid

28. {1-Hydroxy-4-[3-(4-pyridin-4-yl-piperazin-1-yl)-propyl] -cyclohexyl}-acetic acid

29. {1-Hydroxy-4-[5-(4-pyridin-4-yl-piperazin-1-yl)-pentyl] -cyclohexyl}-acetic acid

30. {1-Hydroxy-4-[6-(4-pyridin-4-yl-piperazin-1-yl)-hexyl] -cyclohexyl}-acetic acid

31. {1-Hydroxy-4-[2-oxo-2-(4-pyridin-4-yl-piperazin-1-yl)-ethyl] -cyclohexyl}-acetic acid

32. {1-Hydroxy-4-[3-oxo-3-(4-pyridin-4-yl-piperazin-1-yl)-propyl] -cyclohexyl}-acetic acid

33. {1-Hydroxy-4-[4-oxo-4-(4-pyridin-4-yl-piperazin-1-yl)-butyl] -cyclohexyl}-acetic acid

34. {1-Hydroxy-4-[6-oxo-6-(4-pyridin-4-yl-piperazin-1-yl)-hexyl] -cyclohexyl}-acetic acid

35. {1-Hydroxy-4-[7-oxo-7-(4-pyridin-4-yl-piperazin-1-yl)-heptyl] -cyclohexyl}-acetic acid

36. {4-Hydroxy-1-[2-oxo-2-(4-pyridin-4-yl-piperazin-1-yl)-acetyl] -piperidin-4-yl}-acetic acid

37. 4-Hydroxy-1-[4-oxo-4-(4-pyridin-4-yl-piperazin-1-yl)-butyryl] -piperidin-4-yl}-acetic acid

38. {1-[2,3-Dihydroxy-4-oxo-4-(4-pyridin-4-yl-piperazin-1-yl)-butyryl]-4-hydroxy-piperidin-4-yl}-acetic acid

39. {1-[2,3-Dihydroxy-4-oxo-4-(4-pyridin-4-yl-piperazin-1-yl)-butyryl]-piperidin-4-yl}-acetic acid

40. {4-Hydroxy-1-[7-oxo-7-(4-pyridin-4-yl-piperazin-1-yl)-heptanoyl] -piperidin-4-yl}-acetic acid

41. Hydroxy-[4-(4-pyridin-4-yl-piperazin-1-ylmethyl)-cyclohexyl] -acetic acid

42. Phenyl-[4-(4-pyridin-4-yl-piperazin-1-ylmethyl)-cyclohexyl] -acetic acid

43. Amino-[4-(4-pyridin-4-yl-piperazin-1-ylmethyl)-cyclohexyl] -acetic acid

44. Butane-1-sulfonylamino)-[4-(4-pyridin-4-yl-piperazin-1-ylmethyl)-cyclohexyl]-acetic acid

45. (Butane-1-sulfonylamino)-[l-hydroxy-4-(4-pyridin-4-yl-piperazin-1-ylmethyl)-cyclohexyl]-acetic acid

46. [1-Methoxy-4-(4-pyridin-4-yl-piperazin-1-ylmethyl)-cyclohexyl] -acetic acid

47. (Butane-1-sulfonylamino)-{l-hydroxy-4-[2-(4-pyridin-4-yl-piperazin-1-yl)-ethyl]-cyclohexyl]}-acetic acid

48. (Butane-1-sulfonylamino)-{4-[2-(4-pyridin-4-yl-piperazin-1-yl)-ethyl]-cyclohexyl}-acetic acid

49. 2-[4-(4-Pyridin-4-yl-piperazin-1-ylmethyl)-cyclohexyl] -propionic acid

50. (Butane-1-sulfonylamino-)-{1-hydroxy-4-[2-oxo-2-(4-pyridin-4-yl-piperazin-1-yl)-ethyl]-cyclohexyl}-acetic acid

51. (Butan-1-sulfonylamino-{1-hydroxy-4-[4-oxo-4-(4-pyridin-4-yl-piperazin-1-yl)-butyl]-cyclohexyl}-acetic acid

52. [4-(4-Pyridazin-4-yl-piperazin-1-ylmethyl)-cyclohexyl] -acetic acid

53. [4-(4-pyridazin-4-yl-piperazin-1-carbonyl)-cyclohexyl] -acetic acid

54. {1-[6-(4-Pyridazin-4-yl-piperazin-1-yl)-hexyl]-piperidin-4-yl}-acetic acid

55. {4-[2-Oxo-2-(4-pyridazin-4-yl-piperazin-1-yl)-ethyl] -cyclohexyl}-acetic acid

56. {4-[3-Oxo-3-(4-pyridazin-4-yl-piperazin-1-yl)-propyl] -cyclohexyl}-acetic acid

57. {1-[2-Oxo-2-(4-pyridazin-4-yl-piperazin-1-yl)-acetyl] -piperidin-4-yl}-acetic acid

58. {1-[4-Oxo-4-(4-pyridazin-4-yl-piperazin-1-yl)-butyryl] -piperidin-4-yl}-acetic acid

59. {1-[6-Oxo-6-(4-pyridazin-4-yl-piperazin-1-yl)-hexanoyl] -piperidin-4-yl}-acetic acid

The following examples show several process variants which can be used to synthesize the compounds according to the invention. However, they should not limit the subject matter of the invention. The structure of compounds was established by ¹H and optionally by ¹³C-NMR spectroscopy as well as by mass spectrometry. The purity of the substances was determined by means of C, H, N, and thin layer chromatography.

### EXAMPLE 1

### {4-[2-Oxo-2-(4-pyridin-4-yl-piperazin-1-yl)-ethyl]-cyclohexyl} -acetic acid

1a) 76 g (4-hydroxy-cyclohexyl-)acetic acid ethyl ester produced by catalytic hydrogenation from 4-hydroxyphenyl acetic acid ethyl ester (Raney-Ni/H₂/160° C./200 bar/72 h/ethanol) is dissolved in 130 ml methylene chloride and admixed with 20.6 g Dess-Martin periodinan. The reaction solution is subsequently stirred for 30 h at room temperature. Afterwards the precipitated precipitate is filtered, the methylene chloride solution is washed successively with saturated sodium thiosulfate solution and water and dried over sodium sulfate. After removing the solvent 16.5 g (4-oxocyclohexyl)-acetic acid ethyl ester is obtained as a colourless oil. 1H-NMR (DMSO-d⁶):δ=4.05 ppm (q, 2H); 2.48-2.25 (m, 8 lines, 3H); 2.15 (m, 3H); 1.92 (m, 2H); 1.90 (sextet, 2H), 1.15 (t, 3H).

1b) A solution of 3.6 g (4-oxo-cyclohexyl)-acetic acid ethyl ester and 8 g (tert.-butoxycarbonylmethylene)-triphenylphosphorane (Aldrich GmbH and Co. KG) in 100 ml toluene is heated for 40 hours under reflux. Subsequently the toluene is removed in a vacuum and the product is purified by column chromatography (ethyl acetate/isohexane 1/10). The (4-ethoxycarbonylmethyl-cyclohexylidene)-acetic acid tert.-butyl ester (2.7 g) obtained in this way is dissolved in 40 ml tetrahydrofuran, admixed with 300 mg 10 percent palladium/carbon and hydrogenated for 20 hours at normal pressure and room temperature. Afterwards the catalyst is removed by filtration, the tetrahydrofuran is removed on a rotary evaporator and the residue (2.8 g) is taken up in 5 ml methylene chloride.

After addition of 5 ml trifluoroacetic acid the reaction solution is stirred for 2 h at room temperature and evaporated to dryness. 2.7 g (4-ethoxycarbonylmethyl-cyclohexyl)-acetic acid is obtained in this way. FAB: 228, ¹H-NMR (CDCl₃):δ= 7.70 ppm (broad s, 1H); 4.15 (q, 2H); 2.20 (d, 2H); 2.17 (d, 2H); 1.75 (m, 5H); 1.60 (m, 1H); 1.40 (m, 1H); 1.25 (t, 3H); 1.05 (m, 3H).

1c) A solution of 690 mg (4-ethoxycarbonylmethylcyclohexyl)-acetic acid, 490 mg 1-(4-pyridyl)piperazine and 680 mg dicyclohexylcarbodiimide in 10 ml dimethylformamide is stirred for 48 h at room temperature. Subsequently the dimethylformamide is removed on a rotary evaporator, the residue is taken up in 10 ml water and the aqueous mixture is extracted three times with 10 ml methylene chloride each time. After drying the combined organic phases over sodium sulfate and removing the solvent, the product is purified by means of preparative HPLC (RP-18; methanol/buffer [pH 6.8]=7/3). 770 mg {4-[ 2-oxo-2-(4-pyridin-4-yl-piperazin-1-yl)-ethyl]-cyclohexyl} -acetic acid ethyl ester is obtained. m/e=373; ¹-NMR (DMSO-d⁶): δ=8.18 ppm (d, 2H); 6.82 (d, 2H); 4.05 (q, 2H); 3.60 (broad s, 4H); 3.42 (broad s, 4H); 2.31 (dd, 2H); 2.15 (dd, 2H); 1.55 (m, 5H); 1.45 (m, 1H); 1.40 (m, 1H); 1.15 (t, 3H); 0.95 (broad t, 3H).

1d) A solution of 730 mg of the ethyl ester prepared in 1c) in 6 ml methanol is stirred for 3 h at 50° C. and 2 ml 1 N sodium hydroxide solution. Subsequently the methanol is removed in a vacuum, the residue is taken up in 10 ml water and the product is purified by means of ion chromatography (DOWEX 50 H-form, eluant: aqueous ammonia). 300 mg of the title compound is obtained in this way as a white powder.

FAB: 345; ¹-NMR (CDCl₃):δ=8.20 ppm (d, 2H); 3.60 (broad s, 4H); 3.35 (broad s, 4H); 2.30 (dd, 2H); 2.10 (dd, 2H); 1.90 (m, 1H); 1.65 (m, 4H); 1.55 (m, 1H); 1.40 (m, 1H); 0.95 (m, 3H).

### EXAMPLE 2

### {1-Hydroxy-4-[2-oxo-2-(4-pyridin-4-yl-piperazin-1-yl)-ethyl] -cyclohexyl}-acetic acid

2a) 15.3 g of the ketone prepared in 1a) is dissolved in 200 ml toluene and admixed with 6 ml ethylene glycol and 30 mg p-toluene sulfonic acid. The reaction solution is then heated for 4 h at 140° C. on a water separator, subsequently cooled and washed with 50 ml saturated sodium carbonate solution. Then the aqueous phase is extracted twice with 30 ml methylene chloride each time, the combined organic phases are dried over sodium sulfate and the solvent is evaporated on a rotary evaporator. The ketal which remains (19.3 g) is taken up in 100 ml tetrahydrofuran and the solution is admixed with 100 ml 1 N sodium hydroxide solution. Then the reaction solution is heated for 24 h at 50° C., the tetrahydrofuran is withdrawn in a vacuum, the aqueous solution is acidified with 1 N hydrochloric acid and extracted three times with 50 ml methylene chloride each time. After drying the combined extracts over sodium sulfate and removing the solvent, 13 g (1,4-dioxa-spiro-[ 4,5]dec-8-yl)acetic acid is obtained. FAB: 200.

2b) 11.3 g 2-(1,4-dioxa-spiro[4,5]dec-8-yl)-1-(4-pyridin-4-yl-piperazin-1-yl)-ethanone is obtained analogously to 1c) from 13 g (1,4-dioxa-spiro[4,5]-dec-8-yl)-acetic acid, 10.6 g 1-(4-pyridyl)-piperazine and 15.6 g N-(3-dimethylaminopropyl)-N′-ethyl-carbodiimide hydrochloride. 1H-NMR (CDCl₃): δ =8.25 ppm (d, 2H); 6.58 (d, 2H); 3.38 (s, 4H); 3.70 (t, 2H); 3.58 (t, 2H); 3.30 (q, 4H); 2.25 (d, 2H); 1.85 (m, 1H); 1.70 (m, 4H); 1.52 (sextet, 2H); 1.25 (m, 2H).

2c) A solution of 1.5 g of the product produced in 2b) is stirred for 30 min at room temperature in 10 ml tetrahydrofuran and 5 ml 6 N hydrochloric acid. Subsequently the tetrahydrofuran is removed in a vacuum and the hydrochloric acid solution is adjusted to pH 9 with concentrated ammonia and extracted three times with 5 ml methylene chloride each time. After drying the combined organic phases over sodium sulfate and removing the solvent, the residue is purified by means of column chromatography (mobile solvent: ethyl acetate/methanolic ammonia=9/1). 870 mg 4-[2-oxo-2-(4-pyridin-4-yl-piperazin-1-yl)-ethyl]-cyclohexanone is obtained in this way. ¹H-NMR (CDCl₃): δ=8.35 ppm (d, 2H); 6.65 (d, 2H); 3.85 (broad t, 2H); 3.62 (broad t, 2H); 3.38 (broad q, 4H); 2.40 (m, 7H); 2.25 (m, 2H); 1.45 (m, 2H).

2d) 1.5 ml n-butyllithium (1.6 M in hexane) is added dropwise to a solution of 0.34 ml diisopropylamine in 5 ml tetrahydrofuran stirred at −15° C. under nitrogen, the solution is allowed to stir for a further 30 min at −15° C., subsequently cooled to −78° C. and a solution of 0.27 ml acetic acid tert.-butyl ester in 2 ml tetrahydrofuran is added dropwise. The reaction mixture is now stirred for a further hour at −78° C. and then admixed dropwise with a solution of 600 mg of the cyclohexanone derivative produced in 2c) in 2 ml tetrahydrofuran and 2 ml 1,3-dimethyl-3,4,5,6-tetrahydro-2(1H)-pyrimidione (DMPU). Afterwards the reaction mixture is stirred for a further 30 min at −78° C., subsequently heated to room temperature within one hour and finally stirred for a further three hours at room temperature. After the addition of 5 ml saturated ammonium chloride solution, the reaction mixture is extracted three times with 5 ml methylene chloride each time, the combined organic phases are dried over sodium sulfate, the solvent is removed on a rotary evaporator and the crude product is purified by means of preparative HPLC (Select B, 12μ; methanol/buffer (pH 7.5)=65/35). In this way the two isomers are obtained {1-hydroxy-4-[2-oxo-2-(4-pyridin-4-yl-piperazin-1-yl)-ethyl] -cyclohexyl}-acetic acid tert.-butyl ester (cis/trans isomers).

FAB: 417;

cis-isomer: 340 mg; ¹H-NMR (CDCl₃): δ=8.25 ppm (d, 2H); 6.60 (d, 2H); 3.92 (broad s, 1H; OH); 3.75 (m, 2H); 3.58 (m, 2H); 3.30 (m, 4H); 2.41 (s, 2H); 2.25 (d, 2H); 1.88 (m, 1H); 1.75 (broad t, 4H); 1.50 (t with fine resolution, 2H); 1.40 (s, 9H); 1.08 (broad q, 2H).

trans-isomer: 100 mg; ¹H-NMR (CDCl₃): δ=8.25 ppm (d, 2H); 6.60 (d, 2H); 3.72 (m, 2H); 3.60 (m, 2H); 3.31 (m, 4H); 2.28 (s, 2H); 2.21 (d, 2H); 1.80 (m, 1H); 1.70 (broad d, 2H); 1.55 (m, 2H); 1.40 (s, 9H); 1.34 (m, 4H).

2e) A solution of 300 mg cis-isomer in 1 ml trifluoroacetic acid is stirred for 1 hour at room temperature, it is subsequently evaporated and dried in a high vacuum. In this way 300 mg cis-{1-hydroxy-4-[2-oxo-2-(4-pyridin-4-yl-piperazin-1-yl)-ethyl] -cyclohexyl}-acetic acid trifluoroacetate is obtained as a white powder. m/e: 361.

¹-NMR (DMSO-d⁶): δ=8.29 ppm (d, 2H); 7.20 (d, 2H); 3.65 (broad t, 8H); 2.40 (s, 2H); 2.31 (d, 2H); 1.88-1.60 (m, 5H); 1.35 (broad t, 2H); 1.10 (m, 2H); ¹⁹F-NMR (DMSO-d⁶): δ=−73.30 ppm (s).

2f) 50 mg trans-{1-hydroxy-4-[2-oxo-2-(4-pyridin-4-yl-piperazin-1-yl)-ethyl)-cyclohexyl}-acetic acid-trifluoroacetate is obtained analogously to 2e) from 80 mg trans-isomer. m/e: 361. ¹H-NMR (DMSO-d⁶): δ=8.25 ppm (d, 2H); 7.15 (d, 2H); 3.65 (m, 8H); 2.29 (s, 2H); 2.22 (d, 2H); 1.60 (m, 3H); 1.40 (m, 6H). ¹⁹F-NMR (DMSO-d⁶): δ=−73.30 ppm (s).

### EXAMPLE 3

### {1-Hydroxy-4-[2-(4-pyridin-4-yl-piperazin-1-yl]) -ethyl]-cyclohexyl} -acetic acid

3a) A solution of 12 g 1,4-dioxaspiro[4,5]decan-4-one and 26 g (ethoxycarbonyl-methylene)-triphenylphosphorane in 100 ml toluene is heated for 24 hours at 100° C. Subsequently the toluene is removed in a vacuum, the residue is admixed with 10 ml of a mixture of ethyl acetate/isohexane=1/5, the precipitated triphenylphosphinoxide is removed by filtration, the filtrate is concentrated in a vacuum and the product is purified by column chromatography. 11.4 g (1,4-dioxa-spiro[4,5]-dec-8-ylidene)-acetic acid ethyl ester is obtained as a colourless oil.

m/e: 226

3b) The product (11 g) produced in 3a) is dissolved in 100 ml tetrahydrofuran and the solution is admixed with 1.5 g 10% palladium/carbon. It is then hydrogenated at room temperature and normal pressure until the uptake of hydrogen is completed, it is filtered, evaporated to dryness and the residue is taken up in 50 ml tetrahydrofuran. The solution obtained in this way is added dropwise to a mixture of 1.2 g lithium aluminium hydride in 50 ml tetrahydrofuran in such a way that the reaction temperature does not exceed 50° C. Afterwards the reaction mixture is stirred for a further 2 h at room temperature and subsequently excess lithium aluminium hydride is carefully decomposed with water. After processing the reaction mixture in the usual way, 7.3 g 2-(1,4-dioxa-spiro[ 4,5]dec-8-yl)-ethanol is obtained as a colourless oil. m/e: 186. ¹H-NMR (CDCl₃+CD₃COOD): δ=3.88 ppm (s, 4H); 3.60 (t, 2H); 1.65 (m, 4H); 1.55-1.48 (t overlapped by m, 5H); 1.20 (m, 2H).

3c) 1 g of the alcohol prepared in 3b) is dissolved in 20 ml diethyl ether and admixed with 1.4 ml triethylamine. A solution of 0.47 ml methanesulfonyl chloride in 5 ml diethyl ether is added dropwise to this solution at room temperature. Afterwards the reaction solution is stirred for a further 30 min, it is subsequently washed successively with 10 ml saturated sodium bicarbonate solution and 10 ml water and the organic phase is dried over sodium sulfate. After removing the solvent, the residue is dissolved in 5 ml dimethylformamide. 1 g 1-(4-pyridyl)-piperazine and 0.8 g potassium carbonate is added to this solution, the reaction mixture is heated for 3 h at 50° C. and then the dimethylformamide is removed in a vacuum. The residue is taken up in 5 ml saturated ammonium chloride solution and extracted three times with 5 ml methylene chloride each time. After drying the combined extracts over sodium sulfate and removing the solvent, the crude product is purified by column chromatography (prep. HPLC: Select B, 12μ, methanol/buffer (pH 7.5)=7/3). 0.6 g 1-[2-(1,4-dioxa-spiro[ 4,5]-dec-8-yl)-ethyl]-4-pyridin-4-yl-1-piperazine is obtained in this way as a light grey powder. m/e: 331. ¹-NMR (CDCl₃): δ=8.20 ppm (d, 2H); 6.58 (d, 2H); 3.90 (s, 4H); 3.28 (dd, 4H); 2.48 (t, 4H); 2.35 (t, 2H); 1.67 (broad d, 4H); 1.45 (q, 4H); 1.25 (m, 3H).

3d) 0.8 g 4-[2-(4-pyridin-4-yl-piperazin-1-yl)-ethyl]-cyclohexanone is obtained analogously to 2c) from 1.1 g of the product prepared in 3c). m/e: 287. ¹H-NMR (CDCl₃): δ=8.20 ppm (d, 2H); 6.59 (d, 2H); 3.28 (t, 4H); 2.50 (t, 4H); 2.40 (t, 2H); 2.30 (m, 4H); 2.0 (m, 2H); 1.75 (m, 1H); 1.51-1.26 (m, 4H).

3e) Two stereoisomers {1-hydroxy-4-[2-(4-pyridin-4-yl-piperazin-yl)-ethyl] -cyclohexyl}-acetic acid tert.-butyl ester (cis/trans-isomers) are obtained analogously to 2d) from 580 mg of the product from 3d), 0.27 ml tert.-butyl acetate, 1.5 ml n-butyl-lithium (1.6 M in hexane) and 0.34 ml diisopropylamine.

FAB: 403.

cis-isomer: 190 mg; ¹H-NMR (CDCl₃): δ=8.18 ppm (d, 2H); 6.60 (d, 2H); 3.30 (t, 4H); 2.75 (broad s, 1H; OH); 2.50 (t, 4H); 2.35 (dd, 2H); 2.25 (s, 2H); 1.70 (d, 2H); 1.55-1.35 (s overlapped by m, 9H+ 4H); 1.22 (m, 5H).

trans-isomer: 130 mg; ¹H-NMR (CDCl₃): δ=8.15 ppm (d, 2H); 6.59 (d, 2H); 3.30 (t, 4H); 2.50 (t, 4H); 2.40 (s, 2H); 2.35 (dd, 2H); 2.25 (broad s, 1H; OH); 1.68 (m, 4H); 1.55-1.30 (s covered by m, 9H+ 6H); 1.0 (m, 1H).

3f) 170 mg cis-{1-hydroxy-4-[2-(4-pyridin-4-yl-piperazin-1-yl)-ethyl]-cyclohexyl}-acetic acid trifluoroacetate is obtained analogously to 2e) from 170 mg cis-isomer.

FAB: 347; m.p.: 130° C.

100 mg trans-{1-hydroxy-4-[2-(4-pyridin-4-yl-piperazin-1-yl)-ethyl-cyclohexyl}-acetic acid trifluoroacetate is correspondingly obtained from 110 mg trans-isomer.

FAB: 347; m.p.: 207° C.

### EXAMPLE 4

### {4-[2-(4-Pyridin-4-yl-piperazin-1-yl)-ethyl]-cyclohexyl} -acetic acid

4a) 5.2 g 4-(2-Hydroxy-ethyl)-cyclohexanone is obtained analogously to process 2c) from 9.8 g of product 3b). ¹H-NMR (DMSO-d⁶): δ=4.41 ppm (t, 1H; OH); 3.48 (q, 2H); 2.36 (3 xd, 2H); 2.18 (d with fine resolution, 2H); 1.95 (dt, 2H); 1.85 (m, 1H); 1.40 (q, 2H); 1.30 (m, 2H).

4b) A solution of 5.2 g 4-(2-hydroxy-ethyl)-cyclohexanone, 6.8 g imidazole and 6 g tert.-butyldimethylsilyl chloride in 50 ml dimethylformamide is stirred for 2 h at room temperature. Afterwards the solution is concentrated in a vacuum, the residue is taken up in 10 ml water and the aqueous solution is extracted three times with 10 ml diethyl ether each time. After drying the combined organic phases over sodium sulfate and removing the solvent, the crude product is purified by column chromatography (silica gel, ethyl acetate/isohexane= 1/8). 7 g 4-[2-(tert.-butyl-dimethyl-silanyloxy)-ethyl] -cyclohexanone is obtained in this way as a colourless oil. ¹H-NMR (CDCl₃): δ=3.65 ppm (t, 2H); 2.35 (m, 4H); 2.05 (m, 2H); 1.85 (m, 1H); 1.50 (q, 2H); 1.38 (m, 2H); 0.85 (s, 9H); 0.00 (s, 6H).

4c) 8.1 g {4-[2-(tert.-butyl-dimethyl-silanyloxyethyl] -cyclohexylidene}-acetic acid ethyl ester is obtained analogously to 3a) from 7 g of product 4b) and 9.2 g ethoxycarbonyl-methylene-triphenylphosphorane. ¹H-NMR (CDCl₃): δ=5.52 ppm (s, 1H); 4.10 (q, 2H); 3.70 (broad d, 1H); 3.60 (t, 2H); 2.30-2.05 (m, 2H); 1.85 (m, 3H); 1.60 (m, 1H); 1.40 (q, 2H); 1.20 (t, 3H); 1.06 (m, 2H); 0.85 (s, 9H); 0.00 (s, 6H).

4d) The product 4c) (8 g) is dissolved in 70 ml of a tetrahydrofuran/methanol=1/1 mixture and the solution is admixed with 0.9 g 10% palladium/carbon. It is hydrogenated for 4 h at normal pressure and room temperature, the catalyst is subsequently removed by filtration, the filtrate is evaporated to dryness, the residue is taken up in 70 ml of a 1 M hydrofluoric acid/acetonitrile mixture and the reaction solution is allowed to stir for 12 h at room temperature. Afterwards the solvent is evaporated in a vacuum, the residue is admixed with 10 ml saturated sodium bicarbonate solution and the aqueous solution obtained in this way is extracted three times with 10 ml methylene chloride each time. After drying the combined organic phases over sodium sulfate and chromatographing the residue on silica gel (methylene chloride/methanol=9/1), 2.4 g [4-(2-hydroxy-ethyl)-cyclohexyl] -acetic acid ethyl ester is obtained as a light yellow oil. ¹H-NMR (DMSO-d⁶): δ=4.35 ppm (broad s, 1H; OH); 4.05 (q, 2H); 3.40 (broad s, 2H); 2.15 (sextet, 2H); 1.90 (m, 1H); 1.78-1.25 (m, 8H); 1.16 (t, 3H); 0.90 (m, 3H).

4e) In analogy to process 3c) 2.4 g mesylate is obtained from 2.4 g of product 4d), 0.93 methane sulfonyl chloride and 2.2 ml triethylamine, which yielded 420 mg {4-[2-pyridin-4-yl-piperazin-1-yl)-ethyl] -cyclohexyl}-acetic acid ethyl ester when reacted with 1.4 g 1-(4-pyridyl)-piperazine in the presence of 1.2 g potassium carbonate. FAB: 359; ¹-NMR (CDCl₃): δ=8.20 (d, 2H); 6.55 (d, 2H); 4.08 (q, 2H); 3.25 (t, 4H); 2.50 (t, 4H); 2.35 (m, 2H); 2.0 (d, 2H); 1.68 (m, 5H); 1.46 (m, 2H); 1.35 (m, 2H); 1.19 (t, 3H); 1.13 (m, 1H); 0.90 (t, 2H).

4f) A solution of 170 mg of product 4e) in 4 ml tetrahydrofuran and 0.5 ml water is admixed with 0.57 ml 1 N sodium hydroxide solution. The reaction mixture obtained in this way is heated for 4 h at 50° C. and subsequently the tetrahydrofuran is removed in a vacuum. The residue is then taken up in 5 ml water and the product is purified by means of ion chromatography (Dowex 50 H-Form, eluant: concentrated ammonia). In this way 70 mg of the title compound is obtained as a light grey powder. m/e=331.

### Test Report

Assay

Microtitre plates are coated overnight with 2 μg/ml isolated activated GpIIb/IIIa receptor. After removing the unbound receptor by several washing steps, the surface of the plates is blocked with 1% casein and washed again. The test substance is added in the required concentrations and the plates are incubated for 10 minutes while shaking in a linear shaker. The natural ligand of the gpIIb/IIIa receptor, fibrinogen, is added. After 1 hour of incubation the unbound fibrinogen is removed by several washing steps and bound fibrinogen is detected by a peroxidase-conjugated anti-fibrinogen monoclonal antibody by measuring the O.D. at 405 nm in an ELISA-reader. Inhibition of fibrinogen-GpIIb/IIIa interaction results in a low O.D. An IC₅₀ is calculated in relation to a concentration-effect curve.

Literature

The GpIIb/IIIa-Fibrinogen-ELISA is a modification of the assays described in the following literature:

Nachman, R. L. & Leung, L. L. K. (1982): Complex formation of platelet membrane glycoproteins IIb and IIIa with fibrinogen. J. Clin. Invest. 69:263-269. Wright, P. S. et al. (1993): An echistatin C-terminal peptide activated GpIIbIIIa binding to fibrinogen, fibronectin, vitronectin and collagen type I and type IV. Biochem. J. 293:263-276.

Pharmacological Data

| Example | IC₅₀ (μmol/l) |
|---|---|
| 2 cis-isomer | 0.30 |
| 3 trans-isomer | 0.006 |
| 3 cis-isomer | 0.30 |
| 4 | 0.10 |

Comparative Experiments

The compound cis-1-hydroxy-4-[4-(4-pyridyl)-piperazin-1-yl] -acetic acid was prepared as a reference substance which is included in the patent WO 94/22835 as example No. 102. This compound has an IC₅₀ value of 2.50 μmol/l in the above assay!

1. Compounds of formula I 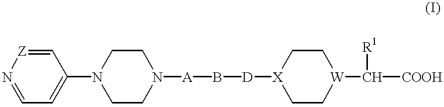
in which
R¹ denotes hydrogen, lower alkyl, lower alkenyl, cycloalkyl, cycloalkenyl, an optionally substituted monocyclic or bicyclic aryl, an optionally substituted hetaryl, an optionally substituted arylalkyl or one of the groups
—OR², —NR³R⁴,
W denotes nitrogen or —CR⁵,
X, Z independently of one another denote nitrogen or the group —CH and in the case that W denotes a nitrogen atom, X must be the —CH group,
A denotes a valency dash or a carbonyl group,
B denotes a valency dash or a C₁-C₆ alkylene chain optionally substituted once or several times by lower alkyl or an OR² group,
D denotes a valency dash and, in the case that X is a nitrogen atom, can also be a carbonyl group in which case A, B and D may not simultaneously denote a valency dash,
R² denotes hydrogen, lower alkyl or arylalkyl,
R³,R⁴ independently of one another denote hydrogen or lower alkyl or together with the nitrogen atom to which they are bound form a five to six-membered heterocyclic ring,
R⁵ denotes hydrogen or a group OR²,
and optical isomers and pharmacologically acceptable salts thereof.

2. Process for the production of compounds of formula I 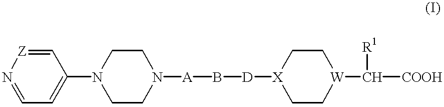
in which
R¹ denotes hydrogen, lower alkyl, lower alkenyl, cycloalkyl, cycloalkenyl, an optionally substituted monocyclic or bicyclic aryl, an optionally substituted hetaryl, an optionally substituted arylalkyl or one of the groups
—OR², —NR³R⁴,
W denotes nitrogen or 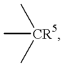
X, Z independently of one another denote nitrogen or the group 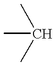
and in the case that W denotes a nitrogen atom, X must be the 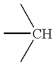
group,
A denotes a valency dash or a carbonyl group,
B denotes a valency dash or a C₁-C₆ alkylene chain optionally substituted once or several times by lower alkyl or an OR² group,
D denotes a valency dash and, in the case that X is a nitrogen atom, can also be a carbonyl group in which case A, B and D may not simultaneously denote a valency dash,
R² denotes hydrogen, lower alkyl or arylalkyl,
R³,R⁴ independently of one another denote hydrogen or lower alkyl or together with the nitrogen atom to which they are bound form a five to six-membered heterocyclic ring,
R⁵ denotes hydrogen or a group OR²
and optical isomers and pharmacologically acceptable salts thereof,
wherein, a compound of formula II 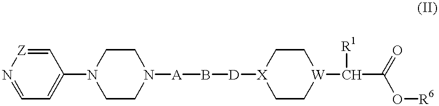
in which R¹, A, B, D, W, X and Z have the meaning stated above and R⁶ denotes a methyl, ethyl, tert.-butyl or benzyl residue is hydrolyzed in a well-known manner and the compounds obtained are converted into their optical isomers and if desired compounds of formula I which are obtained are converted into pharmacologically acceptable salts.

3. Pharmaceutical agents containing at least one compound of formula I as claimed in claim 1 in addition to conventional carriers and auxiliary substances.

4. Use of compounds of formula I as claimed in claim 1 to produce pharmaceutical agents for the treatment of diseases which are a result of thrombo-embolic events.
